(19) United States
(12) Patent Application Publication
(10) Pub. No.: US 2025/0271452 A1
(43) Pub. Date: Aug. 28, 2025

(54) ASSAYS AND METHODS OF USE OF THOSE ASSAYS IN THE CARE AND DIAGNOSIS OF STROKE PATIENTS

(71) Applicants: Matthew Edwardson, Washington, DC (US); Masato Mitsuhashi, Irvine, CA (US); Dennis Van Epps, Irvine, CA (US)

(72) Inventors: Matthew Edwardson, Washington, DC (US); Masato Mitsuhashi, Irvine, CA (US); Dennis Van Epps, Irvine, CA (US)

(21) Appl. No.: 18/584,188

(22) Filed: Feb. 22, 2024

Related U.S. Application Data
(60) Provisional application No. 63/447,539, filed on Feb. 22, 2023.

(51) Int. Cl. G01N 33/68

(52) U.S. Cl. CPC G01N 33/6896; G01N 2333/70596; G01N 2800/2871; G01N 2800/52

(57) ABSTRACT

Extracellular vesicles and/or exosomes (EVs) remain sparsely studied in hemorrhagic transformations, including stroke and intracranial hemorrhages and could provide key insights into stroke pathophysiology. We assessed plasma levels of neuron-derived EVs (NDEs), astrocyte-derived EVs (ADEs), and oligodendrocyte-derived EVs (ODEs) in 58 patients 5, 15, and 30 days post-ischemic stroke along with 46 matched controls using sandwich immunoassays. Hemorrhagic transformation was a better predictor of ADE levels than lesion volume in linear regression models. These findings suggest that ADE levels are preferentially increased over the first month post-stroke compared to EVs from other neural cell types. ADEs may be of additional interest as biomarkers of blood-brain barrier breakdown to predict hemorrhagic transformation at earlier time points after stroke.

This United States Utility Application claims priority to United States Provisional Patent Application Ser. No.: 63447539 entitled “Assays for Use in the Care and Diagnosis of Stroke Patients”, which was filed on Feb. 22, 2023 and which is commonly owned and incorporated herein in its entirety by reference.

### GOVERNMENT RIGHTS

This invention was made with government support under KL2TR001432 and UL1TR001409 awarded by the National Institutes of Health. The government has certain rights in the invention.

### FIELD OF THE SUBJECT MATTER

The field of the subject matter is assays and methods of use of those assays in the care and diagnosis of stroke patients.

### BACKGROUND

Extracellular vesicles or exosomes (herein interchangeable and referred to as EVs, herein) are small vesicles (<150 nm-1000 nm) with a lipid bilayer produced by nearly all cell types [1]. EVs contain proteins, lipids, and RNAs [1]. Their primary role was once thought to be cellular waste removal, but EVs are increasingly implicated in intercellular communication [2]. For example, EVs derived from astrocyte cultures led to neurite outgrowth in a rodent model of spinal cord injury [3]. EVs can cross the blood-brain barrier [4]. Investigators have isolated EVs of presumed neural, astrocytic or oligodendrocytic origin using immunoassays for their respective cell surface proteins [5]. This has sparked great interest in isolating brain-derived EVs (BDEs) from the peripheral blood to characterize physiologic changes in the CNS that are otherwise not easily accessible in living humans.

BDEs are increasingly reported as biomarkers in neurological disease, but few have studied them in clinical stroke. Plasma neuron-derived EVs (NDEs) were shown to contain increased levels of pathologic proteins in Alzheimer's patients compared to controls and in some cases even predict progression to dementia in healthy elderly adults [6-8]. Plasma astrocyte-derived EVs (ADEs) were decreased after traumatic brain injury, but contained dramatically increased complement levels compared to controls [9]. In ischemic stroke, total EV levels increase acutely in serum and may promote inflammation [10]. Looking more specifically at BDEs, only one study was identified related to clinical stroke. It was discovered that low NDE levels around 5-45 days after stroke were associated with worse outcomes on the Barthel Index at 6-months [11]. Preclinically, most studies suggest BDEs play a role in neural repair after ischemic injury [12-15].

BDEs may also serve as biomarkers of damage to the blood-brain barrier (BBB) after stroke. From a theoretical perspective, one would expect small vesicles to more easily pass from the CNS to the peripheral blood in the setting of BBB breakdown. There are 2 main peaks of BBB permeability after stroke, the first around 6-12 hours related to acute hypoxic injury and the second around 2-4 days from neuroinflammation [16, 17]. From 1 week to 1 month post-stroke increased permeability continues due to small leaky blood vessels created in the process of angiogenesis [17]. Most hemorrhagic transformation of ischemic stroke takes place during the second peak and occurs in around 9% of patients [18]. Patients with hemorrhagic transformation have significantly greater morbidity and mortality [18, 19]. As it pertains to BDEs, a prior study showed increased NDE levels immediately after aortic arch replacement surgery that subsided over time and correlated with post-operative delirium [20]. No studies have directly assessed BDEs in the setting of ischemic stroke with hemorrhagic transformation.

### SUMMARY OF THE SUBJECT MATTER

Contemplated embodiments, as disclosed herein, include generally assays for use in the care and diagnosis of stroke patients.

Additionally, embodiments disclosed herein include: 1) identification and support for the identification of hemorrhagic conversion in ischemic stroke patients; 2) use of contemplated assays to track changes in plasma EV as an indicator of the status of blood brain barrier (BBB) post stroke; 3) monitoring the impact of treatment on blood EV as an indicator of recovery of BBB in stroke, and 4) an indicator of intracranial hemorrhage (ICH).

Generally, methods of analyzing a sample from a subject comprise the steps of: obtaining a biological sample comprising vesicles from a subject; isolating vesicles from the biological sample; and detecting one or more biomarkers from the isolated vesicles, wherein at least one of the one or more biomarkers comprise CD9, L1CAM, CD171, EAAT1, EAAT2, and MOG.

Specifically, methods of analyzing a sample from a subject comprise the steps of: obtaining a biological sample comprising at least one brain-derived vesicle from the subject, measuring the level of one or more biomarkers in the biological sample, and comparing the level of the one or more biomarkers in the biological sample to a control level of the one or more biomarkers in a control biological sample.

In addition, methods of analyzing a sample from a subject to determine hemorrhagic transformation comprise the steps of: obtaining a biological sample comprising at least one brain-derived vesicle from the subject, measuring the level of one or more biomarkers in the biological sample, and comparing the level of the one or more biomarkers in the biological sample to a control level of the one or more biomarkers in a control biological sample, wherein an increase in the one or more biomarkers in the biological sample as compared to the control level of the one or more biomarkers in a control biological sample is an indicator of hemorrhagic transformation or brain hemorrhage after a stroke in a patient. As used herein, hemorrhagic transformation comprises brain hemorrhage, intracranial hemorrhage, or a combination thereof.

Also, methods of analyzing a sample from a subject to determine hemorrhagic transformation, comprising the steps of: obtaining a biological sample comprising at least one vesicle from the subject, measuring the level of the at least one vesicle in the biological sample, and comparing the level of the at least one vesicle in the biological sample to a control level of the at least one vesicle in a control biological sample, wherein an increase in the at least one vesicle in the biological sample as compared to the control level of the at least one vesicle in a control biological sample is an indicator of hemorrhagic transformation after a stroke in a patient.

Post-stroke blood-brain barrier assays are contemplated herein that include the steps of: obtaining a biological sample comprising at least one brain-derived vesicle from the subject, measuring the level of one or more biomarkers in the biological sample, and comparing the level of the one or more biomarkers in the biological sample to a control level of the one or more biomarkers in a control biological sample.

Post-stroke blood-brain barrier assays are contemplated herein that include the steps of obtaining a biological sample comprising at least one brain-derived vesicle from the subject, measuring the level of one or more biomarkers in the biological sample, and comparing the level of the one or more biomarkers in the biological sample to a control level of the one or more biomarkers in a control biological sample, wherein an increase in the one or more biomarkers in the biological sample as compared to the control level of the one or more biomarkers in a control biological sample is an indicator of hemorrhagic transformation or brain hemorrhage after a stroke in a patient.

Post-stroke blood-brain barrier assays are also contemplated that include the steps of obtaining a biological sample comprising at least one vesicle from the subject, measuring the level of the at least one vesicle in the biological sample, and comparing the level of the at least one vesicle in the biological sample to a control level of the at least one vesicle in a control biological sample, wherein an increase in the at least one vesicle in the biological sample as compared to the control level of the at least one vesicle in a control biological sample is an indicator of hemorrhagic transformation after a stroke in a patient.

Also, methods of analyzing a sample from a subject to determine hemorrhagic transformation comprise the steps of: obtaining a biological sample comprising at least one brain-derived vesicle from the subject, measuring the level of one or more biomarkers in the biological sample, preparing a control biological sample that comprises brain-derived vesicles; and comparing the level of the one or more biomarkers in the biological sample to a control level of the one or more biomarkers in the control biological sample, wherein an increase in the one or more biomarkers in the biological sample as compared to the control level of the one or more biomarkers in a control biological sample is an indicator of hemorrhagic transformation after a stroke in a patient.

In addition, methods of analyzing a sample from a subject to determine hemorrhagic transformation comprise the steps of: obtaining a biological sample comprising at least one brain-derived vesicle from the subject, measuring the level of one or more biomarkers in the biological sample, preparing a control biological sample that comprises neural cell type vesicles; and comparing the level of the one or more biomarkers in the biological sample to a control level of the one or more biomarkers in the control biological sample, wherein an increase in the one or more biomarkers in the biological sample as compared to the control level of the one or more biomarkers in a control biological sample is an indicator of hemorrhagic transformation or ICH after a stroke in a patient.

### BRIEF DESCRIPTION OF THE FIGURES

FIG. 1. Elevation of extracellular vesicle (EV) concentrations in ischemic stroke versus control participants for (A) neuron-derived, (B) astrocyte-derived, and (C) oligodendrocyte-derived EVs (mean±SE). There were no significant differences between the controls from day 0 to day 30. Wherever n<46 for control or n<58 for ischemic stroke there were samples that returned no measurable result. *p<0.05; **p<0.01.

FIG. 2. Extracellular vesicle (EV) concentrations (mean±SE) in ischemic stroke patients with stroke volume <10 mL versus >10 mL for (A) neuron-derived, (B) astrocyte-derived, and (C) oligodendrocyte-derived EVs. Wherever n<37 for volume <10 mL or n<21 for volume >10mL there were samples that returned no measurable result. Astrocyte-derived EV concentrations for individual participants (bar represents mean) at (D) 5 days, (E) 15 days, and (F) 30 days post-stroke. *p<0.05.

FIG. 3. Extracellular vesicle (EV) concentrations (mean ±SE) in ischemic stroke patients with and without hemorrhagic transformation for (A) neuron-derived, (B) astrocyte-derived, and (C) oligodendrocyte-derived EVs. Wherever n<46 for hemorrhagic transformation (−) there were samples that returned no measurable result. Astrocyte-derived EV (ADE) concentrations for individual participants (bar represents mean) at (D) 5 days, (E) 15 days, and (F) 30 days post-stroke. The 3 outliers with the highest ADE levels 15 days post-stroke are labeled in red, green, and blue to show how these outliers fluctuated over time. *p<0.05.

FIG. 4. Astrocyte-derived extracellular vesicle concentrations 5 days post-stroke versus ischemic stroke lesion volume, including designation of which participants had hemorrhagic transformation (triangles) and those with no hemorrhage (circles). Stroke lesion volume and hemorrhagic transformation were closely related, as evidenced by a chi-squared test with p=0.002.

FIG. 5. Intravenous tPA status for ischemic stroke participants and their corresponding extracellular vesicle (EV) concentrations (mean±SE) at 5, 15, and 30 days post-stroke for (a) neuron-derived, (b) astrocyte-derived, and (c) oligodendrocyte-derived EVs. Wherever n<42 for no IV-tPA there were samples that returned no measurable result. NDE at 5, 15, and 30 days respectively: p=0.69, p=0.74, p=0.6; ADE at 5, 15, and 30 days respectively: p=0.4, p=0.91, p=0.52; ODE at 5, 15, and 30 days respectively: p=0.45, p=0.47, and p=0.39.

FIG. 6. Mechanical thrombectomy status for ischemic stroke patients and their corresponding extracellular vesicle (EV) concentrations (mean±SE) at 5, 15, and 30-days post-stroke for (a) neuron-derived, (b) astrocyte-derived, and (c) oligodendrocyte-derived EVs. Wherever n<51 for no thrombectomy there were samples that returned no measurable result. Astrocyte-derived EV (ADE) concentrations for individual participants (bar represents mean) at (d) 5 days, (e) 15 days, and (f) 30 days post-stroke. *p<0.05.

FIG. 7. TOAST classification categorizing the ischemic stroke etiology for participants and their corresponding extracellular vesicle (EV) concentrations (mean±SE) 5, 15, and 30-days post-stroke for (a) neuron-derived, (b) astrocyte-derived, and (c) oligodendrocyte-derived EVs. Wherever n<20 for lacunar, n<11 for undetermined, or n<10 for large artery there were samples that returned no measurable result. Undetermined=stroke of undetermined etiology. Only 3 participants had stroke of other determined etiology, so these were excluded.

### DETAILED DESCRIPTION

Contemplated embodiments, as disclosed herein, include generally assays for use in the care and diagnosis of stroke patients.

Additionally, embodiments disclosed herein include: 1) identification and support for the identification of hemorrhagic conversion in ischemic stroke patients; 2) use of contemplated assays to track changes in plasma EV as an indicator of the status of blood brain barrier (BBB) post stroke; 3) monitoring the impact of treatment on blood EV as an indicator of recovery of BBB in stroke, and 4) an indicator of intracranial and/or brain hemorrhage (ICH). As used herein, the phrase “intracranial hemorrhage” refers to any bleeding within the intracranial vault, including the brain parenchyma and surrounding meningeal spaces.

Generally, methods of analyzing a sample from a subject comprise the steps of: obtaining a biological sample comprising vesicles from a subject; isolating vesicles from the biological sample; and detecting one or more biomarkers from the isolated vesicles, wherein at least one of the one or more biomarkers comprise CD9, L1CAM, CD171, EAAT1, EAAT2, and MOG.

Specifically, methods of analyzing a sample from a subject comprise the steps of: obtaining a biological sample comprising at least one brain-derived vesicle from the subject, measuring the level of one or more biomarkers in the biological sample, and comparing the level of the one or more biomarkers in the biological sample to a control level of the one or more biomarkers in a control biological sample, wherein at least one of the one or more biomarkers comprise CD9, L1CAM, CD171, EAAT1, EAAT2, and MOG.

In addition, methods of analyzing a sample from a subject to determine hemorrhagic transformation comprise the steps of: obtaining a biological sample comprising at least one brain-derived vesicle from the subject, measuring the level of one or more biomarkers in the biological sample, and comparing the level of the one or more biomarkers in the biological sample to a control level of the one or more biomarkers in a control biological sample, wherein at least one of the one or more biomarkers comprise CD9, L1CAM, CD171, EAAT1, EAAT2, and MOG, wherein an increase in the one or more biomarkers in the biological sample as compared to the control level of the one or more biomarkers in a control biological sample is an indicator of hemorrhagic transformation after a stroke in a patient. As used herein, hemorrhagic transformation comprises brain hemorrhage, intracranial hemorrhage (ICH), or a combination thereof.

Also, methods of analyzing a sample from a subject to determine hemorrhagic transformation, comprising the steps of: obtaining a biological sample comprising at least one vesicle from the subject, measuring the level of the at least one vesicle in the biological sample, and comparing the level of the at least one vesicle in the biological sample to a control level of the at least one vesicle in a control biological sample, wherein an increase in the at least one vesicle in the biological sample as compared to the control level of the at least one vesicle in a control biological sample is an indicator of hemorrhagic transformation after a stroke in a patient.

Post-stroke blood-brain barrier assays are contemplated herein that include the steps of: obtaining a biological sample comprising at least one brain-derived vesicle from the subject, measuring the level of one or more biomarkers in the biological sample, and comparing the level of the one or more biomarkers in the biological sample to a control level of the one or more biomarkers in a control biological sample, wherein at least one of the one or more biomarkers comprise CD9, L1CAM, CD171, EAAT1, EAAT2, and MOG.

Post-stroke blood-brain barrier assays are contemplated herein that include the steps of obtaining a biological sample comprising at least one brain-derived vesicle from the subject, measuring the level of one or more biomarkers in the biological sample, and comparing the level of the one or more biomarkers in the biological sample to a control level of the one or more biomarkers in a control biological sample, wherein at least one of the one or more biomarkers comprise CD9, L1CAM, CD171, EAAT1, EAAT2, and MOG, wherein an increase in the one or more biomarkers in the biological sample as compared to the control level of the one or more biomarkers in a control biological sample is an indicator of hemorrhagic transformation and ICH after a stroke in a patient.

Post-stroke blood-brain barrier assays are also contemplated that include the steps of obtaining a biological sample comprising at least one vesicle from the subject, measuring the level of the at least one vesicle in the biological sample, and comparing the level of the at least one vesicle in the biological sample to a control level of the at least one vesicle in a control biological sample, wherein an increase in the at least one vesicle in the biological sample as compared to the control level of the at least one vesicle in a control biological sample is an indicator of hemorrhagic transformation after a stroke in a patient.

Also, methods of analyzing a sample from a subject to determine hemorrhagic transformation or ICH comprise the steps of: obtaining a biological sample comprising at least one brain-derived vesicle from the subject, measuring the level of one or more biomarkers in the biological sample, preparing a control biological sample that comprises brain-derived vesicles; and comparing the level of the one or more biomarkers in the biological sample to a control level of the one or more biomarkers in the control biological sample, wherein at least one of the one or more biomarkers comprise CD9, L1CAM, CD171, EAAT1, EAAT2, and MOG, wherein an increase in the one or more biomarkers in the biological sample as compared to the control level of the one or more biomarkers in a control biological sample is an indicator of hemorrhagic transformation or ICH after a stroke in a patient.

In addition, methods of analyzing a sample from a subject to determine hemorrhagic transformation or ICH comprise the steps of: obtaining a biological sample comprising at least one brain-derived vesicle from the subject, measuring the level of one or more biomarkers in the biological sample, preparing a control biological sample that comprises neural cell type vesicles; and comparing the level of the one or more biomarkers in the biological sample to a control level of the one or more biomarkers in the control biological sample, wherein at least one of the one or more biomarkers comprise CD9, L1CAM, CD171, EAAT1, EAAT2, and MOG, wherein an increase in the one or more biomarkers in the biological sample as compared to the control level of the one or more biomarkers in a control biological sample is an indicator of hemorrhagic transformation or ICH after a stroke in a patient.

It is to be understood that the contemplated embodiments are not limited to the particular methodologies, protocols, cell lines, assays, and reagents described herein, as these may vary. It is also to be understood that the terminology used herein is intended to describe particular embodiments of the contemplated embodiments and is in no way intended to limit the scope of these embodiments as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments; a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which the contemplated embodiments disclosed herein belong. Although any methods and materials similar or equivalent to those described herein can be used in the practice or testing of the contemplated embodiments, the preferred methods, devices, and materials are now described. All publications cited herein are incorporated herein by reference in their entirety for the purpose of describing and disclosing the methodologies, reagents, and tools reported in the publications that might be used in connection with the contemplated embodiments. Nothing herein is to be construed as an admission that the contemplated embodiments are not entitled to antedate such disclosure by virtue of any prior patent applications, patents, or disclosures.

The practice of the contemplated embodiments herein will utilize, unless otherwise indicated, conventional methods of chemistry, biochemistry, molecular biology, cell biology, genetics, immunology and pharmacology, within the skill of the art. Such techniques are explained fully in the literature. See, e.g., Gennaro, A.R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D.M. Weir and C.C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The contemplated embodiments relate, in part, to the discovery that exosomal biomarkers can be assayed to identify subjects who have had a stroke or who may be likely to have a stroke.

Current contemplated embodiments are based, in part, on the discovery of unexpected increases in certain biomarkers in brain-derived exosomes present in the circulation of subjects who have had or may have a stroke. Current embodiments demonstrate that exosomal levels of these biomarkers may be assayed to assist diagnosis of a stroke or the intensity of a stroke. Current embodiments further show that measurement of certain biomarkers in brain-derived exosomes, as compared to neural-derived exosomes, from a subject may be used to predict a stroke or the likelihood of a stroke or ICH in a patient.

Contemplated embodiments also provide compositions for use in the methods described herein. Such compositions may include small molecule compounds; peptides and proteins including antibodies or functionally active fragments thereof; and polynucleotides including small interfering ribonucleic acids (siRNAs), micro-RNAs (miRNAs), ribozymes, and anti-sense sequences. (See, e.g., Zeng (2003) Proc Natl Acad Sci USA 100:9779-9784; and Kurreck (2003) Eur J Biochem 270:1628-1644.)

The section headings are used herein for organizational purposes only and are not to be construed as in any way limiting the subject matter described herein.

### Biological Sample

Current contemplated embodiments provide biomarkers and diagnostic and prognostic methods for identifying strokes in a patient. Contemplated embodiments also provide biomarkers for quantifying exosome levels in biological samples. Biomarkers and exosome levels are determined in a biological sample obtained from a subject. In some embodiments, the biological sample of contemplated embodiments can be obtained from blood. In some embodiments, about 0.25-10 mL of blood is drawn from a subject. In other embodiments, about 10-50 mL of blood is drawn from a subject. Blood can be drawn from any suitable area of the body, including an arm, a leg, or blood accessible through a central venous catheter. In some embodiments, blood is collected following a treatment or activity. For example, blood can be collected following a medical exam. The timing of collection can also be coordinated to increase the number and/or composition of exosomes present in the sample. For example, blood can be collected following exercise or a treatment that induces vascular dilation.

Blood may be combined with various components following collection to preserve or prepare samples for subsequent techniques. For example, in some embodiments, blood is treated with an anticoagulant, a cell fixative, a protease inhibitor, a phosphatase inhibitor, a protein, a DNA, or an RNA preservative following collection. In some embodiments, blood is collected via venipuncture using vacuum collection tubes containing an anticoagulant such as EDTA or heparin. Blood can also be collected using a heparin-coated syringe and hypodermic needle. Blood can also be combined with components that will be useful for cell culture. For example, in some embodiments, blood is combined with cell culture media or supplemented cell culture media (e.g., cytokines).

Biological samples can also be obtained from other sources known in the art, including whole blood, serum, plasma, urine, interstitial fluid, peritoneal fluid, cervical swab, tears, saliva, buccal swab, skin, cerebrospinal fluid, or other tissues including, for example, brain tissues.

### Enrichment or Isolation of Exosomes

Samples can be enriched for exosomes through positive selection, negative selection, or a combination of positive and negative selection. In some embodiments, exosomes are directly captured. In other embodiments, blood cells are captured and exosomes are collected from the remaining biological samples. In some embodiments, the exosomes enriched in the biological samples are neuron-derived exosomes, astrocyte-derived exosomes, oliogodendrocyte-derived exosomes, and microglia-derived exosomes.

Samples can also be enriched for exosomes based on differences in the biochemical properties of exosomes. For example, samples can be enriched for exosomes based on antigen, size, electrophoretic separation, nucleic acid, metabolic, gene expression, or epigenetic differences. In some of the embodiments based on antigen differences, antibody-conjugated magnetic or paramagnetic beads in magnetic field gradients or fluorescently labeled antibodies with flow cytometry are used. In some of the embodiments based on nucleic acid differences, flow cytometry is used. In some of the embodiments based on metabolic differences, dye uptake/exclusion measured by flow cytometry or another sorting technology is used. In some of the embodiments based on gene expression, cell culture with cytokines is used. Samples can also be enriched for exosomes based on other biochemical properties known in the art. For example, samples can be enriched for exosomes based on pH or motility. Further, in some embodiments, more than one method is used to enrich for exosomes. In other embodiments, samples are enriched for exosomes using antibodies, ligands, or soluble receptors.

In other embodiments, surface markers are used to positively enrich exosomes in the sample. In other embodiments, CD9, L1CAM, CD171, EAAT1, EAAT2, and MOG are used to enrich for exosomes. In some embodiments, cell surface markers that are not found on exosomes populations are used to negatively enrich exosomes by depleting cell populations. Flow cytometry sorting may also be used to further enrich for exosomes using cell surface markers or intracellular or extracellular markers conjugated to fluorescent labels. Intracellular and extracellular markers may include nuclear stains or antibodies against intracellular or extracellular proteins preferentially expressed in exosomes. Cell surface markers may include antibodies against cell surface antigens that are preferentially expressed on exosomes (e.g., NCAM). In some embodiments, the cell surface marker is a neuron-derived exosome surface marker, including, for example, CD9, L1CAM, CD171, EAAT1, EAAT2, and MOG. In some embodiments, CD9, L1CAM, CD171, EAAT1, EAAT2, and MOG antibody or other antibodies directed toward astrocyte, neuron, or oligodendrocyte selective markers used to enrich or isolate exosomes from the sample. In certain aspects, the CD9, L1CAM, CD171, EAAT1, and MOG antibody is biotinylated. In this embodiment, biotinylated CD171 antibody can form an antibody-exosome complex that can be subsequently isolated using streptavidin-agarose resin or beads. In other embodiments, the CD9, L1CAM, CD171, EAAT1, EAAT2, and MOG antibody is a monoclonal anti-human CD9, L1CAM, CD171, EAAT1, EAAT2, and MOG antibody.

In some embodiments, enriched exosomes from the biological sample are subsequently enriched for a specific type of exosome (e.g., a subpopulation of exosomes). For example, the biological sample is enriched for exosomes and then the enriched exosomes are subsequently enriched for neural-derived exosomes. In some embodiments, the biological sample is enriched for individual neural cell sources of exosomes. In certain aspects, the neural cell sources of exosomes are microglia, oligodendrocytes, neurons, or astrocytes. In other embodiments, surface markers are used to enrich for a specific type of exosome (e.g., neural-derived exosome). In some embodiments, CD9, L1CAM, CD171, EAAT1, EAAT2, and/or MOG cell surface markers are used to enrich for a specific type of exosome. In some embodiments, cell surface markers that are not found on the exosomes of interest are used to negatively enrich exosomes by depleting unwanted cell populations. Flow cytometry sorting may also be used to further enrich for specific types of exosomes using cell surface markers or intracellular or extracellular markers conjugated to fluorescent labels. Intracellular and extracellular markers may include nuclear stains or antibodies against intracellular or extracellular proteins preferentially expressed in or on the exosomes of interest. Cell surface markers may include antibodies against cell surface antigens that are preferentially expressed on exosomes (e.g., CD9, L1CAM, CD171, EAAT1, EAAT2, and MOG). In some embodiments, the cell surface marker is a brain-derived exosome surface marker. In some embodiments, the cell surface marker is an astrocyte-derived exosome surface marker, including, for example, EAAT1 or EAAT2. In some embodiments, the cell surface marker is an oligodendrocyte-derived exosome surface marker, including, for example, MOG. In some embodiments the cell surface marker is a receptor for dopamine, serotonin, GABA, glutamate, opioid, orexin, adrenalin, noradrenalin, acetylcholine, and/or dopamine transporter. In some embodiments, a monoclonal EAAT1 or MOG antibody is used to enrich or isolate exosomes from the sample. In certain aspects, the CD9, L1CAM, CD171, EAAT1, EAAT2, and MOG antibody is biotinylated. In this embodiment, biotinylated CD9, L1CAM, CD171, EAAT1, EAAT2, and MOG antibody can form an antibody-exosome complex that can be subsequently isolated using streptavidin-agarose resin or beads. In other embodiments, the CD9, L1CAM, CD171, EAAT1, EAAT2, and MOG antibody is a monoclonal anti-human CD9, L1CAM, CD171, EAAT1, EAAT2, and MOG antibody.

In other embodiments, exosomes are isolated or enriched from a biological sample comprising: contacting a biological sample with an agent under conditions wherein an exosome present in said biological sample binds to said agent to form an exosome-agent complex; and isolating said exosome from said exosome-agent complex to obtain a sample containing said exosome, wherein the purity of exosomes present in said sample is greater than the purity of exosomes present in said biological sample. In certain embodiments, the agent is an antibody or a lectin. Lectins useful for forming an exosome-lectin complex are described in U.S. Patent Application Publication No. 2012/0077263.

In some embodiments, the exosomes are neuron-derived exosomes, astrocyte-derived exosomes, oliogodendrocyte-derived exosomes, or microglia-derived exosomes. In some embodiments, multiple isolating or enriching steps are performed. In certain aspects of the present embodiment, a first isolating step is performed to isolate exosomes from a blood sample and a second isolating step is performed to isolate neural-derived exosomes from other exosomes. In other embodiments, the exosome portion of the exosome-agent complex is lysed using a lysis reagent and the protein levels of the lysed exosome are assayed. In some embodiments, the antibody-exosome complex is created on a solid phase. In yet other embodiments, the methods further comprise releasing the exosome from the antibody-exosome complex. In certain embodiments, the solid phase is non-magnetic beads, magnetic beads, agarose, or sepharose. In other embodiments, the exosome is released by exposing the antibody-exosome complex to low pH between 3.5 and 1.5. In yet other embodiments, the released exosome is neutralized by adding a high pH solution. In other embodiments, the released exosome is lysed by incubating the released exosomes with a lysis solution. In still other embodiments, the lysis solution contains inhibitors for proteases and phosphatases.

### Stroke and Stroke-Related Disorders

Contemplated embodiments provide methods for diagnosing or prognosing a stroke in a subject, identifying a subject at risk of a stroke, or prescribing a therapeutic regimen or predicting benefit from therapy in a subject having a stroke.

In some contemplated embodiments embodiments, a medical practitioner is enabled to assist or support diagnosis or prognosis of a stroke in a subject. In other contemplated embodiments, a medical practitioner is enabled to rule out or eliminate a stroke as a diagnostic possibility. In yet other contemplated embodiments, a medical practitioner is enabled to identify a subject at risk of having a stroke. In other contemplated embodiments, a medical practitioner is enabled to predict whether a subject will later have a stroke or ICH. In further contemplated embodiments, a medical practitioner is enabled to prescribe a therapeutic regimen or predict benefit from therapy in a subject having a stroke or ICH.

### Biomarkers

Biomarker levels are assayed in a biological sample obtained from a subject having or at-risk of having a stroke or ICH. In some embodiments, the biomarker is a receptor for dopamine, serotonin, GABA, glutamate, opioid, orexin, adrenalin, noradrenalin, acetylcholine, synaptic proteins, and/or dopamine transporter, GAP43, neurofilament light chain, Neuroligin or other selective markers for types of BDE. In some embodiments, the biomarker is CD9, L1CAM, CD171, EAAT1, EAAT2, and MOG. Other known brain-derived biomarkers may be used in combination with the biomarkers of the contemplated embodiments, as well as other markers that result in the identification of NDE, ADE, or ODE that CD9, L1CAM, CD171, EAAT1, EAAT2, and MOG. Still other examples of biomarkers are provided in US Patent Application Pub. No. 2015/0119278, the contents of which are hereby incorporated by reference.

In some embodiments, contemplated biomarker levels are measured by determining the quantity or gene expression of the biomarker. In certain embodiments, gene expression changes are measured by determining the expression level of one or more of the genes. In certain aspects, gene expression of the biomarker is determined using PCR, microarray, or sequencing. In some embodiments, the expression level of the biomarker is determined by measuring the mRNA or miRNA level of the biomarker.

One of ordinary skill in the art has several methods and devices available for the detection and analysis of contemplated markers. With regard to polypeptides or proteins in patient test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassay (RIAs), competitive binding assays, planar waveguide technology, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated and disclosed herein. The antibodies could be immobilized onto a variety of solid supports, such as magnetic or chromatographic matrix particles, the surface of an assay place (such as microtiter wells), pieces of a solid substrate material (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of markers may be carried out separately or simultaneously with one test sample. Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvageable tissue, the appropriateness of drug therapies, the effectiveness of various therapies, identification of the severity of the event, identification of the disease severity, and identification of the patient's outcome, including risk of future events.

An assay consisting of a combination of the markers contemplated herein may be constructed to provide relevant information related to differential diagnosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out methods described within contemplated embodiments disclosed herein to optimize clinical sensitivity or specificity in various clinical settings.

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different analytes. Such formats include protein microarrays, or “protein chips” and capillary devices.

Biomarkers of contemplated embodiments serve an important role in the early detection and monitoring of strokes and stroke-related disorders. Markers of such disorders are typically substances found in a bodily sample that can be measured. The measured amount can correlate to underlying disorder or disease pathophysiology, presence or absence of a stroke or stroke-related disorder, probability of a stroke or stroke-related disorder in the future. In patients receiving treatment for their condition the measured amount will also correlate with responsiveness to therapy. In some embodiments, an increase in the level of one or more biomarkers of contemplated embodiments disclosed herein is indicative of a stroke or stroke-related disorder. Accordingly, the methods of contemplated embodiments disclosed herein are useful for the differential diagnosis of a stroke or stroke-related disorder.

In some embodiments, the biomarker is measured by a method selected from the group consisting of immunohistochemistry, immunocytochemistry, immunofluorescence, immunoprecipitation, western blotting, and ELISA.

The work herein includes an assessment of levels of NDEs, ADEs, and oligodendrocyte-derived EVs (ODEs) over the first month post-stroke. The period from 5-30 days after ischemic stroke represents an important transition from CNS injury to repair. It was postulated that BDE levels would evolve over time, with primarily NDEs and ADEs elevated early after ischemia, and ODEs elevated late during the process of remyelination. We further hypothesized that all BDE types would be elevated early in patients who experienced hemorrhagic transformation compared to those without hemorrhage.

A principal finding from this study was that plasma astrocyte-derived extracellular vesicles (ADEs) remained significantly elevated 5-30 days post-ischemic stroke. We also found weak associations between ADE levels and stroke lesion size, ADE levels and hemorrhagic transformation, and ADE levels and mechanical thrombectomy. These findings could shed light on post-stroke physiology.

The most consistent finding in this study was that ADE levels remained significantly elevated from 5-30 days post-ischemic stroke. The neural-and oligodendrocyte-derived EV levels appeared slightly increased, but with this sample size were no different than matched controls statistically. One possible explanation could be that all brain cells produce more EVs after stroke and astrocytes simply outnumber other cell types in the human brain allowing us to find a statistically significant difference for ADEs in this study of modest sample size. A long held misconception in the field suggested a 10:1 ratio of glial to neural tissue in the human brain²³, but this was later shown to be true only in white matter²⁴. In addition, oligodendrocytes outnumber astrocytes throughout the brain in a 3:1 ratio²⁴. Therefore, a more likely explanation for our findings is that stroke preferentially induces the release of ADEs over vesicles from other cell types. Indeed, ADEs were shown to increase by 34% in rodent brain tissue 24 hours post-stroke whereas NDEs, ODEs, and microglial EVs remained unchanged¹⁵. Prior human studies demonstrated an increase in serum or plasma EVs post-stroke, but none looked specifically at ADEs^{10,25}.

Astrocytes undergo hypertrophy with elongated processes within minutes of cerebral ischemia and form a glial scar within days, presumably to provide trophic support to injured neurons and prevent the spread of ischemic cell death^{26,27}. Reactive astrogliosis may last for months to years depending on the severity of the injury²⁸. This timeframe for astrocyte activation is in line with our finding of increased ADEs from 5-30 days post-stroke. Some have shown evidence that ADEs can provide neural protection or combat reactive oxygen species in neuronal cell lines subjected to hypoxic ischemic conditions^{29,30}. Over the first month post-stroke the brain is likely transitioning from neural injury to repair. ADE secretion during this timeframe could be due to ongoing neurotrophic support to injured neurons or the promotion of neural repair. For example, one study found evidence that ADEs subjected to oxygen glucose deprivation and treated with a semaphorin 3A inhibitor promoted axonal outgrowth in ischemic neurons¹⁶.

We expected the concentration of all brain-derived EVs to increase in plasma in the setting of hemorrhagic transformation since hemorrhage is associated with breakdown of the blood-brain barrier, but only found an association with ADE populations. A simple explanation might be that because ischemia induces a preferential increase in ADEs they are more likely to pass through a damaged BBB. An alternative explanation could stem from the close proximity of astrocytes to endothelial cells in the neurovascular unit. Astrocytic end feet surround the endothelial cells and help maintain the integrity of the BBB¹⁸. In the setting of ischemia both astrocytes and endothelial cells have impaired integrin signaling, contributing to breakdown of the BBB³¹. It follows that ADEs would preferentially enter the plasma over other brain-derived EVs in the setting of BBB disruption.

Prior studies have shown, and we demonstrated here, that hemorrhagic transformation is closely tied to stroke lesion size^{19,32}. Our findings suggest that lesion size is more closely related to hemorrhagic transformation than ADE levels. It remains possible, however, that brain-derived EVs levels in the hyperacute phase (<24 hours post-stroke) or specific proteins within EVs may prove to be stronger biomarkers of hemorrhagic transformation.

We also found increased ADE levels at 5 and 30-days post-stroke in patients who received mechanical thrombectomy. Five of the 7 patients who received thrombectomy had moderate levels of hemorrhagic transformation (HI-2 or PH-1). Bleeding into the infarct bed is common after thrombectomy, though most studies only report the rate of severe hemorrhagic conversion (PH-2) which was 5.1% in a pooled analysis of thrombectomy trials³³. We suspect that increased ADE levels following mechanical thrombectomy were related to breakdown of the blood-brain barrier due to reperfusion of ischemic endothelial cells.

We found a weak association between an increase in ADEs and hemorrhagic transformation at specific time points post-stroke driven by 2-3 outliers with very high levels. A diagnostic test with a high cutoff for ruling in major risk of hemorrhagic transformation (test with high specificity) could be helpful to choose which patients to avoid treating with thrombolytics. The current study was limited in that we had no hyperacute (<24 hours) plasma samples. In addition, we had no patients who experienced the most severe degree of hemorrhagic transformation, PH-2, which leads to significant clinical deterioration and in some cases death. Such a diagnostic test would have to be accurate in the hyperacute phase and predict these symptomatic hemorrhages to hold clinical utility.

We assume the EV populations to be of CNS origin because the sandwich immunoassay technique will only measure L1CAM⁺/CD9⁺ double positive ELISA signals as an NDEs. Free floating L1CAM or CD9⁺ EV's lacking L1CAM would not be detected with this assay. Similarly, only EAAT1⁺/CD9⁺ and MOG⁺/CD9⁺ double positive signals would be measured as ADEs and ODEs respectively.

We found that ADEs were preferentially increased over other brain-derived EV types in stroke patients versus controls from 5-30 days post-stroke. Exciting future studies await to fully investigate brain-derived EV proteins, RNAs, and lipids after stroke. This will provide much greater insight into the physiologic transition from brain injury to repair post-stroke and help determine how EVs can be viable biomarkers for clinical events such as hemorrhagic transformation and ICH.

### EXAMPLES

### Study Participants

Ischemic stroke participants were recruited prospectively between 2014 and 2020 from the Biomarkers of Stroke Recovery Study (BIOREC, MedStar Georgetown University Hospital, Washington, DC). The study protocol was approved by the local IRB and carried out according to their guidelines and regulations (Georgetown University IRB #2015-0288). BIOREC was a longitudinal observational study that collected blood plasma 5, 15, and 30 days after stroke in patients with mild, moderate, or severe arm motor impairment. Forty-six participants completed all assessments including a 90-day follow up and 12 participants completed assessments only through 30 days.

Control participants were recruited prospectively under the BIOREC protocol using a HIPPA waiver to screen the electronic health record (EHR) followed by full consent prior to sample collection. We used a computer algorithm to screen all patients in the MedStar EHR and identify near perfectly matched controls for the previously enrolled stroke participants based on age, race, ethnicity, sex, and cardiovascular comorbidities including hypertension, diabetes, atrial fibrillation, smoking, and statin use [21]. Only the 46 BIOREC participants who completed all study assessments had a matched control.

### Plasma Collection and Storage

Fasting blood samples were collected in the morning by venipuncture using EDTA tubes (Cardinal Health, OH, USA). Samples were placed on ice, delivered to the biorepository, and centrifuged at 2600 RPM for 10 min at 20° C. Platelet-poor plasma was carefully removed with a pipette without disturbing the buffy coat and frozen at −80° C. All sample processing was performed within approximately 4 hours from sample collection.

### Neuroimaging

All participants had a clinical MRI as part of their routine care that was assessed for lesion volume and presence and degree of hemorrhagic transformation (ME). Lesion volume was estimated using the ABC/2 method [22]. The degree of hemorrhagic transformation was graded according to the ECASS criteria adapted for MRI [23]. The 4 types of hemorrhagic transformation in order of increasing severity include: hemorrhagic infarct type 1 (HI-1, small petechial hemorrhage), hemorrhagic infarct type 2 (HI-2, confluent petechial hemorrhage), parenchymal hematoma type 1 (PH-1, hematoma <⅓ of infarct bed), and parenchymal hematoma type 2 (PH-2, hematoma >⅓ of infarct bed).

### Sandwich Immunoassay

Mouse monoclonal antibody against human L1CAM, CD171 (Thermo Fisher Scientific, Waltham, MA), EAAT1 (Abcam, Cambridge, UK), MOG (Thermo Fisher), and CD9 (BD Biosciences, San Jose, CA) were utilized as indicators of NDEs, ADEs, ODEs and a common exosome membrane marker respectively. Control mouse IgG2a was obtained from BioLegend (San Diego, CA), and normal mouse IgG was obtained from Equitech-Bio (Kerrville, TX).

Antibodies used for immobilization (anti-CD171, anti-EAAT1, anti-MOG and control mouse IgG2a and purified normal mouse IgG were diluted in ELISA coating buffer (BioLegend) in a final concentration of 2.5 μg/mL, and 50 μL was applied to ELISA wells (Corning #3923, Sigma Aldrich, St Louis MO). After 1-hour incubation, each well was washed once with phosphate buffered saline (PBS, Thermo Fisher), and incubated with undiluted blocker casein (Thermo Fisher) for another 1 hour. After each well was washed twice with PBS, ELISA wells were stored in a refrigerator.

The protocol for the double label sandwich ELISA immunoassay in this study is similar to a previous publication [20 ] which demonstrated the size (100-200 nm, consistent with small EVs) and electron micrographic characterization of NDEs using this method. In the current study, EV data represent the double label of the CD171 NDEs or EAAT1 ADEs or MOG ODEs with the CD9 common EV marker. In brief, 40 μL samples of plasma were applied to ELISA wells and incubated for 1 hour at room temperature. After the first washing step, each well was reacted with the CD9 biotinylated probes (50 ng/mL) supplemented with 0.8% bovine serum albumin (BSA, Thermo Fisher), 40 μg/mL mouse IgG (Equitech-Bio) for another 1 hour. Biotinylation was carried out by EZ link Sulfo-NHS-LC-Biotin (Thermo Fisher) followed by the spin column procedure to remove free biotin. After the second washing step, each well was reacted with a 1/4,000 dilution of poly-horseradish peroxidase (HRP)-conjugated streptavidin (Thermo Fisher) supplemented with 10% BSA (Equitech-Bio) and 30% blocker casein (Thermo Fisher), and incubation was continued for 20 min. After the third washing step, each well was incubated for 5 min with 0.0006% H₂O₂ (CVS pharmacy, Irvine, CA) diluted in PBS: water (1:1) solution to remove non-specifically bound HRP conjugates. After aspiration of H₂O₂, each well was mixed with ⅓ dilution of chemiluminescent substrate (Super Signal, Thermo Fisher) for 4 min, then relative light units (RLU) were determined by a luminometer (Active GLO, ANSH Labs, Webster, TX). Using a standard plasma, arbitrarily assigned to 100 units/mL (U/mL). ELISA readings of RLU (Relative Light Units) were converted to U/mL by a 4-parameter logistic formula.

### Statistical Analysis

Two-tailed Student's t-tests were used to determine whether NDE, ADE, or ODE levels were significantly different between stroke and control participants and between participants with and without hemorrhagic transformation at all time points studied. Single factor ANOVAs were performed with TOAST classification as the independent variable and BDE levels at each respective time point as the dependent variable. Stroke of other determined etiology was excluded from ANOVA analysis due to only 3 participants in this category. Chi-squared test was performed using SPSS version 28.

### Results

### Participant Characteristics

The timing of the study visits for blood sample collection and functional outcome measures are shown in Table 1 for the 58 ischemic stroke participants. Twelve participants did not complete the 90-day follow up assessment. The stroke participants had moderate to severe disability at day 5 (mean modified Rankin Scale =4.3 +0.7) that improved over the course of 90 days. The stroke and control groups were well-matched with regard to age, sex, race, ethnicity, and cardiovascular comorbidities with the exception of hyperlipidemia (Table 2). Twenty-eight percent of stroke participants received TPA and 12% received thrombectomy. The TOAST classifications were as follows: 34% lacunar, 24% cardioembolic, 19% undetermined etiology, 17% large artery atherosclerosis, and 5% other determined etiology.

### Extracellular Vesicle Levels in Ischemic Stroke Participants

In order to determine whether stroke affected the levels of NDEs, ADEs and ODEs, the quantities of these vesicles in the stroke (n=58) and control (n=46) populations (FIG. 1) were compared. A small number of the samples were either missing or the assay returned no measurable result, so these were treated as missing data. We found that the level of ADEs were significantly elevated in stroke participants compared to controls 5, 15, and 30 days post-stroke (mean ±SE at 5 days:149.9±18.8 U/mL vs. 83.6±7.5 U/mL, p=0.002; 15 days:149.8±17.7 U/mL vs. 83.6±7.5 U/mL, p=0.002; 30 days:138±15.9 U/mL vs. 83.6±7.5 U/mL, p=0.005). The increase for the NDEs and ODEs did not reach the level of significance (NDE vs. control at 5, 15, and 30 days respectively: p=0.11, p=0.15, p=0.11; ODE vs. control at 5, 15, and 30 days respectively: p=0.32, p=0.38, and p=0.35). The levels of NDE, ADE and ODE were also resampled in the control population 30 days after the original samples were drawn and no differences were found over time for each of the 3 EV types (NDE: p=0.91; ADE: p=0.62; ODE: p=0.95). In summary, ADEs were the only vesicle type significantly elevated 5-30 days post-stroke and there were no significant changes over time among the controls.

### Extracellular Vesicle Levels and Lesion Size

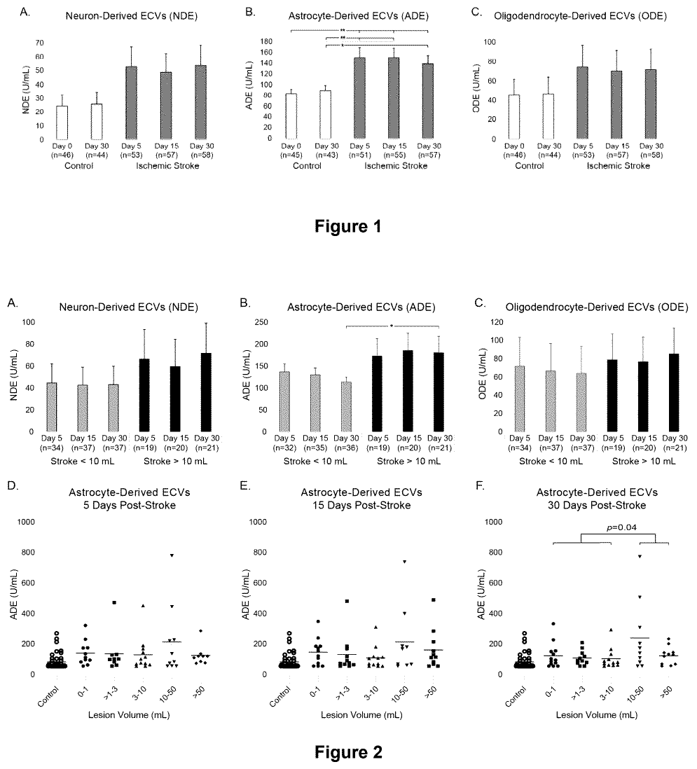

We next determined whether NDE, ADE, or ODE levels were associated with stroke lesion size. The mean stroke lesion size±SE among all participants was 22.8±5.1 mL (range 0.2-156.8 mL). We separated the participants into those with lesions >10 mL (n=21) and those with lesions <10mL (n=37) thinking that lesions >10 mL would reflect more significant brain injury and plotted NDE, ADE and ODE levels over time (FIG. 2). There was a significant increase in ADEs in those with lesions >10 mL compared to those with lesions <10 mL, but only at 30 days post-stroke (mean±SE at 5 days:172.5±40.3 U/mL vs. 136.5±18.2 U/mL, p=0.36; 15 days:185.2±39.2 U/mL vs. 129.6±16 U/mL, p=0.13; 30 days:180.1±37.4 U/mL vs. 113.4±11.2 U/mL, p=0.04). The increase for the NDEs and ODEs for lesions >10 mL vs. <10 mL did not reach the level of significance (NDE at 5, 15, and 30 days respectively: p=0.48, p=0.56, p=0.35; ODE at 5, 15, and 30 days respectively: p=0.88, p=0.82, and p=0.64). Thus, there were only weak associations between the number of brain-derived EVs and lesion size.

### Extracellular Vesicle Levels and Hemorrhagic Transformation

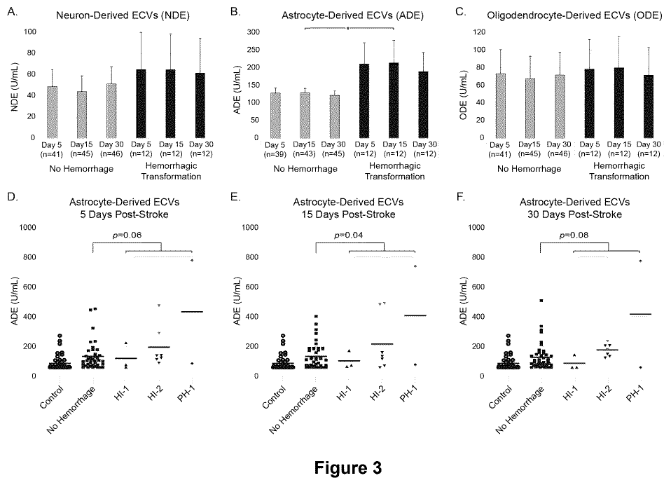

We subsequently assessed whether the number of NDEs, ADEs or ODEs were associated with hemorrhagic transformation (FIG. 3). Twelve participants were confirmed to have hemorrhagic transformation on neuroimaging (3 with HI-1, 7 with HI-2, and 2 with PH-1). There was a significant increase in ADEs in those with hemorrhagic transformation at day 15 as well as trends toward an increase in ADEs at days 5 and 30 (mean±SE at 5 days: 214±61.4 U/mL vs. 130.2±15.1 U/mL, p=0.06; 15 days:217.6±64.7 U/mL vs. 130.9±13 U/mL, p=0.04; 30 days:191.6.1±55.5 U/mL vs. 123.7±13.3 U/mL, p=0.08). There were no significant differences in levels of NDEs or ODEs between participants with and without hemorrhagic transformation (NDE at 5, 15, and 30 days respectively: p=0.65, p=0.54, p=0.78; ODE at 5, 15, and 30 days respectively: p=0.93, p=0.82, and p=1.0). To summarize, hemorrhagic transformation was associated with an increase in ADE levels 15 days post-stroke and trends toward an increase at 5 and 30 days post-stroke but was not significantly associated with NDE and ODE levels.

### Relationship Between Lesion Size and Hemorrhagic Transformation

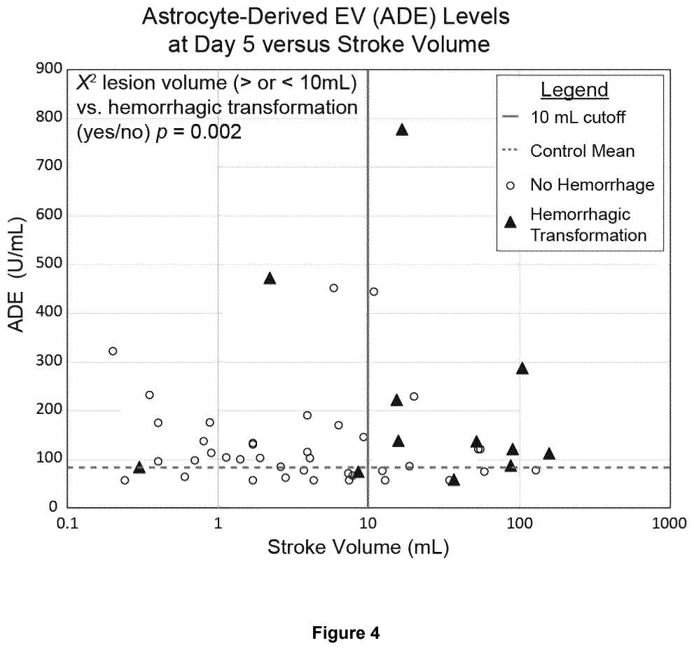

Finally, we determined whether lesion size and hemorrhagic transformation were related and their respective influence on ADE levels. The mean lesion size±SE for those with and without hemorrhagic transformation was 48.8±14.4 mL and 16±4.8 mL respectively (FIG. 4). A chi-squared test using lesion size greater or less than 10 mL suggested that lesion size and hemorrhagic transformation were closely related (X²(1,58) =9.9, p=0.002). Multiple linear regression was used to test if hemorrhagic transformation (yes/no) or lesion size predicted ADE levels at 5 days post-stroke. The fitted regression model was: ADE level (U/mL)=140.5±113.5*(hemorrhagic transformation 0 or 1 where 1=yes)−0.82*(lesion size in mL). The overall regression was not statistically significant (R²=0.11, F(2,48)=2.9, p=0.063). However, hemorrhagic transformation significantly predicted ADE levels in the model (β=113.5, p=0.02) whereas lesion size did not (β=−0.82, p=0.16). Similar regression models testing whether hemorrhagic transformation and lesion size predict ADE levels at 15- and 30-days post-stroke were not significant overall (p=0.13 and 0.14 respectively). These additional models showed a trend toward hemorrhagic transformation predicting ADE levels whereas lesion size did not (15-days post-stroke: hemorrhagic transformation (β=87.8, p=0.054), lesion size (β=−0.03, p=0.94; 30-days post-stroke: hemorrhagic transformation (β=81.1, p=0.051), lesion size (β=−0.41, p=0.35). In summary, there was a strong relationship between hemorrhagic transformation and lesion size. In addition, hemorrhagic transformation was a better predictor of ADE levels than lesion size 5 days post-stroke with similar trends up to 30 days.

A principal finding from this study was that plasma astrocyte-derived extracellular vesicles (ADEs) remained significantly elevated 5-30 days post-ischemic stroke. We also found a weak association between ADE levels and stroke lesion size and a stronger association between ADE levels and hemorrhagic transformation. These findings could shed light on post-stroke physiology. In addition, ADE levels may be of interest to try and predict hemorrhagic transformation at earlier time points post-stroke.

### Elevation in Astrocyte-Derived EV's 5-30 Days Post-Stroke

The most consistent finding in this study was that ADE levels remained significantly elevated from 5-30 days post-ischemic stroke. The neural-and oligodendrocyte-derived EV levels appeared slightly increased, but with this sample size were no different than matched controls statistically. One possible explanation would be that all brain cells produce more EVs after stroke and astrocytes simply outnumber other cell types in the human brain allowing us to find a statistically significant difference for ADEs in this study of modest sample size. There was a long held misconception in the field suggesting a 10:1 ratio of glial to neural tissue in the human brain [24], but this is only true in white matter [25]. In addition, oligodendrocytes outnumber astrocytes throughout the brain in a 3:1 ratio [25]. Therefore, a more likely explanation for our findings is that stroke preferentially induces the release of ADEs over vesicles from other cell types. Indeed, ADEs were shown to increase by 34% in rodent brain tissue 24 hours post-stroke whereas NDEs, ODEs, and microglial EVs remained unchanged [14]. Prior human studies demonstrated an increase in serum or plasma EVs post-stroke, but none looked specifically at ADEs [10, 26].

Astrocytes undergo hypertrophy with elongated processes within minutes of cerebral ischemia and form a glial scar within days, presumably to provide trophic support to injured neurons and prevent the spread of ischemic cell death [27]. Some have shown evidence that ADEs can provide neural protection or combat reactive oxygen species in neuronal cell lines subjected to hypoxic ischemic conditions [28, 29]. From 5-30 days after stroke the brain is likely transitioning from neural injury to repair. ADE secretion during this timeframe could be due to ongoing neurotrophic support to injured neurons or the promotion of neural repair. For example, one study found evidence that ADEs subjected to oxygen glucose deprivation and treated with a semaphorin 3A inhibitor promoted axonal outgrowth in ischemic neurons [15].

### Extracellular Vesicle Levels and tPA, Thrombectomy, and TOAST Classification

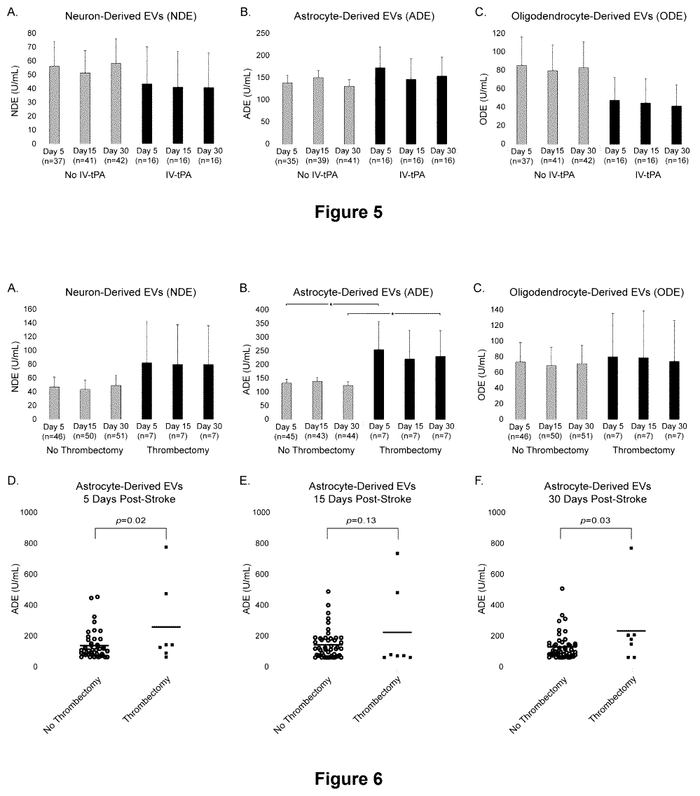

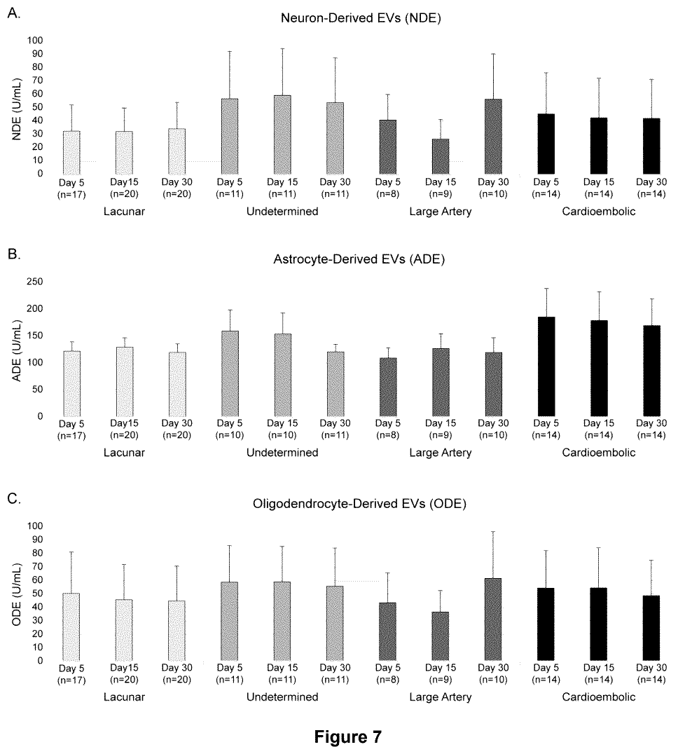

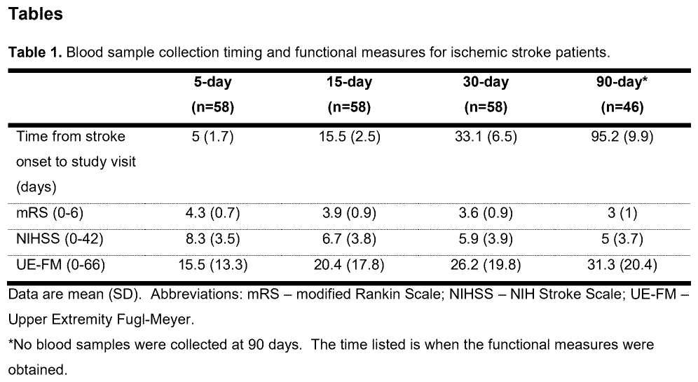

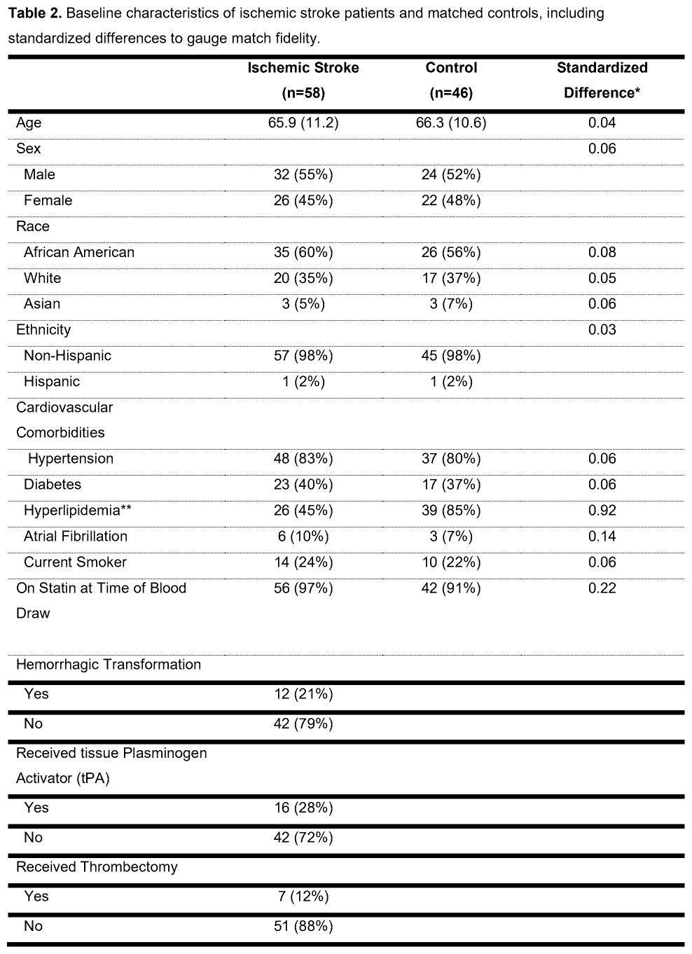

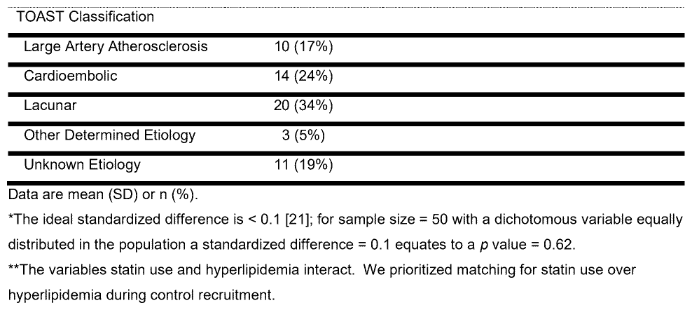

In our final analysis we sought to determine whether EV levels were dysregulated in patients receiving IV-tPA (Alteplase), mechanical thrombectomy, or with strokes of different etiologies. There were no significant differences in levels of NDEs, ADEs or ODEs between participants who did or did not receive IV-tPA (FIG. 5) (NDE at 5, 15,and 30 days respectively: p=0.69, p=0.74, p=0.6; ADE at 5, 15, and 30 days respectively: p=0.4, p=0.91, p=0.52; ODE at 5, 15, and 30 days respectively: p=0.45, p=0.47, and p=0.39). There was a significant increase in ADEs in those who received mechanical thrombectomy at day 5 and 30 compared to those who did not receive thrombectomy (FIG. 6; mean±SE at 5 days:254.9±101.7 U/mL vs. 133.2±14.1 U/mL, p=0.02; 15 days:220.7±101.7 U/mL vs. 139.5±13.8 U/mL, p=0.13; 30 days: 230.3±93.3 U/mL vs. 125±12.3 U/mL, p=0.03). There were no significant differences in levels of NDEs or ODEs between participants who did or did not receive thrombectomy (NDE at 5, 15, and 30 days respectively: p=0.42, p=0.38, p=0.5; ODE at 5, 15, and 30 days respectively: p=0.92, p=0.88, and p=0.97). Five out of 7 patients who underwent thrombectomy had hemorrhagic conversion, 3 with HI-2 and 2 with PH-1. The mean lesion volume in participants receiving thrombectomy was 21.8 mL. There were no differences in EV levels between strokes of different etiologies according to the TOAST classification for each type of BDE at each respective time point (FIG. 7; ANOVA NDE at 5, 15, and 30 days respectively: p=0.93, p=0.85, p=0.93; ADE at 5, 15, and 30 days respectively: p=0.46, p=0.48, p=0.31, ODE at 5, 15, and 30 days respectively: p=0.99, p=0.96, and p=0.98). In summary, mechanical thrombectomy was associated with increased ADE levels at certain time points post-stroke which may be related to the high rate of hemorrhagic transformation in this population.

### Astrocyte-Derived EV Levels and Hemorrhagic Transformation

We expected the concentration of all brain-derived EVs to increase in plasma in the setting of hemorrhagic transformation since hemorrhage is associated with breakdown of the blood-brain barrier, but only found this to be true for ADE populations. A simple explanation might be that because ischemia induces a preferential increase in ADEs they are more likely to pass through a damaged BBB. An alternative explanation could stem from the close proximity of astrocytes to endothelial cells in the neurovascular unit. Astrocytic end feet surround the endothelial cells and help maintain the integrity of the BBB. In the setting of ischemia both astrocytes and endothelial cells have impaired integrin signaling, contributing to breakdown of the BBB [30]. It follows that ADEs would preferentially enter the plasma over other brain-derived EVs in the setting of BBB disruption.

Prior studies have shown, and we demonstrated here, that hemorrhagic transformation is closely tied to stroke lesion size [18, 31]. Our findings raised the question of whether ADE levels simply increase in relation to stroke lesion size or whether they are disproportionately elevated in patients with hemorrhagic transformation. We found only a weak association between ADE levels and lesion size, but a more consistent association between ADE levels and hemorrhagic transformation, indicating ADEs are disproportionately elevated in the setting of hemorrhage. This may again relate to the key role of astrocytes in the neurovascular unit and their active involvement in BBB disruption.

### REFERENCES

1. Shah R, Patel T, Freedman JE. Circulating Extracellular Vesicles in Human Disease. The New England journal of medicine. 2018;379(10):958-66. doi:10.1056/NEJMra1704286.
2. Budnik V, Ruiz-Canada C, Wendler F. Extracellular vesicles round off communication in the nervous system. Nature reviews Neuroscience. 2016;17(3):160-72. doi:10.1038/nrn.2015.29.
3. Sun H, Cao X, Gong A, Huang Y, Xu Y, Zhang J, et al. Extracellular vesicles derived from astrocytes facilitated neurite elongation by activating the Hippo pathway. Exp Cell Res. 2022;411(1):112937. doi:10.1016/j.yexcr.2021.112937.
4. Zhuang X, Xiang X, Grizzle W, Sun D, Zhang S, Axtell RC, et al. Treatment of brain inflammatory diseases by delivering exosome encapsulated anti-inflammatory drugs from the nasal region to the brain. Mol Ther. 2011;19(10):1769-79. doi:10.1038/mt.2011.164.
5. Ohmichi T, Mitsuhashi M, Tatebe H, Kasai T, Ali El-Agnaf OM, Tokuda T. Quantification of brain-derived extracellular vesicles in plasma as a biomarker to diagnose Parkinson's and related diseases. Parkinsonism Relat Disord. 2019;61:82-7. doi:10.1016/j.parkreldis.2018.11.021.
6. Fiandaca MS, Kapogiannis D, Mapstone M, Boxer A, Eitan E, Schwartz JB, et al. Identification of preclinical Alzheimer's disease by a profile of pathogenic proteins in neurally derived blood exosomes: A case-control study. Alzheimers Dement. 2015;11(6):600-7 e1. doi:10.1016/j.jalz.2014.06.008.
7. Goetzl EJ, Boxer A, Schwartz JB, Abner EL, Petersen RC, Miller BL, et al. Altered lysosomal proteins in neural-derived plasma exosomes in preclinical Alzheimer disease. Neurology. 2015;85(1):40-7. doi:10.1212/WNL.0000000000001702.
8. Kapogiannis D, Mustapic M, Shardell MD, Berkowitz ST, Diehl TC, Spangler RD, et al. Association of Extracellular Vesicle Biomarkers With Alzheimer Disease in the Baltimore Longitudinal Study of Aging. JAMA neurology. 2019;76(11):1340-51. doi:10.1001/jamaneurol.2019.2462.
9. Goetzl EJ, Yaffe K, Peltz CB, Ledreux A, Gorgens K, Davidson B, et al. Traumatic brain injury increases plasma astrocyte-derived exosome levels of neurotoxic complement proteins. FASEB J. 2020;34(2):3359-66. doi:10.1096/fj.201902842R.
10. Couch Y, Akbar N, Davis S, Fischer R, Dickens AM, Neuhaus AA, et al. Inflammatory Stroke Extracellular Vesicles Induce Macrophage Activation. Stroke; a journal of cerebral circulation. 2017;48(8):2292-6. doi: 10.1161/STROKEAHA.117.017236.
11. Kowalski, R. G. et al. Rapid Activation of Neuroinflammation in Stroke: Plasma and Extracellular Vesicles Obtained on a Mobile Stroke Unit. Stroke; a journal of cerebral circulation, doi:10.1161/STROKEAHA.122.041422 (2023).
12. Jodicke RA, Huo S, Krankel N, Piper SK, Ebinger M, Landmesser U, et al. The Dynamic of Extracellular Vesicles in Patients With Subacute Stroke: Results of the “Biomarkers and Perfusion-Training-Induced Changes After Stroke” (BAPTISe) Study. Front Neurol. 2021;12:731013. doi:10.3389/fneur.2021.731013.
13. Wu W, Liu J, Yang C, Xu Z, Huang J, Lin J. Astrocyte-derived exosome-transported microRNA-34c is neuroprotective against cerebral ischemia/reperfusion injury via TLR7 and the NF-kB/MAPK pathways. Brain Res Bull. 2020;163:84-94. doi:10.1016/j.brainresbull.2020.07.013.
14. Xin H, Wang F, Li Y, Lu QE, Cheung WL, Zhang Y, et al. Secondary Release of Exosomes From Astrocytes Contributes to the Increase in Neural Plasticity and Improvement of Functional Recovery After Stroke in Rats Treated With Exosomes Harvested From MicroRNA 133b-Overexpressing Multipotent Mesenchymal Stromal Cells. Cell Transplant. 2017;26(2):243-57. doi: 10.3727/096368916X693031.
15. Brenna S, Altmeppen HC, Mohammadi B, Rissiek B, Schlink F, Ludewig P, et al. Characterization of brain-derived extracellular vesicles reveals changes in cellular origin after stroke and enrichment of the prion protein with a potential role in cellular uptake. J Extracell Vesicles. 2020;9(1):1809065. doi:10.1080/20013078.2020.1809065.
16. Hira K, Ueno Y, Tanaka R, Miyamoto N, Yamashiro K, Inaba T, et al. Astrocyte-Derived Exosomes Treated With a Semaphorin 3A Inhibitor Enhance Stroke Recovery via Prostaglandin D2 Synthase. Stroke; a journal of cerebral circulation. 2018;49(10):2483-94. doi:10.1161/STROKEAHA.118.021272.
17. Yang Y, Rosenberg GA. Blood-brain barrier breakdown in acute and chronic cerebrovascular disease. Stroke; a journal of cerebral circulation. 2011;42(11):3323-8. doi:10.1161/STROKEAHA. 110.608257.
18. Bernardo-Castro S, Sousa JA, Bras A, Cecilia C, Rodrigues B, Almendra L, et al. Pathophysiology of Blood-Brain Barrier Permeability Throughout the Different Stages of Ischemic Stroke and Its Implication on Hemorrhagic Transformation and Recovery. Front Neurol. 2020;11:594672. doi:10.3389/fneur.2020.594672.
19. Paciaroni M, Agnelli G, Corea F, Ageno W, Alberti A, Lanari A, et al. Early hemorrhagic transformation of brain infarction: rate, predictive factors, and influence on clinical outcome: results of a prospective multicenter study. Stroke; a journal of cerebral circulation. 2008;39(8):2249-56. doi:10.1161/STROKEAHA.107.510321.
20. The NINDS t-PA Stroke Study Group. Intracerebral hemorrhage after intravenous t-PA therapy for ischemic stroke. Stroke; a journal of cerebral circulation. 1997;28(11):2109-18. doi:10.1161/01.str.28.11.2109.
21. Hotta N, Tadokoro T, Henry J, Koga D, Kawata K, Ishida H, et al. Monitoring of Post-Brain Injuries By Measuring Plasma Levels of Neuron-Derived Extracellular Vesicles. Biomark Insights. 2022;17:11772719221128145. doi:10.1177/11772719221128145.
22. Edwardson MA, Fernandez SJ. Recruiting Control Participants into Stroke Biomarker Studies. Translational stroke research. 2020;11(5):861-70. doi:10.1007/s12975-020-00780-6.
23. Kandel ER, Schwartz JH, Jessel TM, Siegelbaum SA, Hudspeth AJ. Principles of Neural Science. 5th ed. New York: McGraw Hill Professional; 2012.
24. Herculano-Houzel S. The glia/neuron ratio: how it varies uniformly across brain structures and species and what that means for brain physiology and evolution. Glia. 2014;62(9):1377-91. doi:10.1002/glia.22683.
25. Simak J, Gelderman MP, Yu H, Wright V, Baird AE. Circulating endothelial microparticles in acute ischemic stroke: a link to severity, lesion volume and outcome. J Thromb Haemost. 2006;4(6):1296-302. doi:10.1111/j.1538-7836.2006.01911.x.
26. Liu Z, Chopp M. Astrocytes, therapeutic targets for neuroprotection and neurorestoration in ischemic stroke. Prog Neurobiol. 2016;144:103-20. doi:10.1016/j.pneurobio.2015.09.008.
27. Sims, N. R. & Yew, W. P. Reactive astrogliosis in stroke: Contributions of astrocytes to recovery of neurological function. Neurochem Int 107, 88-103, doi:10.1016/j.neuint.2016.12.016 (2017).
28. Li, L. et al. The Specific Role of Reactive Astrocytes in Stroke. Front Cell Neurosci 16, 850866, doi:10.3389/fncel.2022.850866 (2022).
29. Guitart K, Loers G, Buck F, Bork U, Schachner M, Kleene R. Improvement of neuronal cell survival by astrocyte-derived exosomes under hypoxic and ischemic conditions depends on prion protein. Glia. 2016;64(6):896-910. doi:10.1002/glia.22963.
30. Pascua-Maestro R, Gonzalez E, Lillo C, Ganfornina MD, Falcon-Perez JM, Sanchez D. Extracellular Vesicles Secreted by Astroglial Cells Transport Apolipoprotein D to Neurons and Mediate Neuronal Survival Upon Oxidative Stress. Front Cell Neurosci. 2018;12:526. doi:10.3389/fncel.2018.00526.
31. del Zoppo GJ. The neurovascular unit in the setting of stroke. J Intern Med. 2010;267(2):156-71. doi:10.1111/j.1365-2796.2009.02199.x.
32. Marsh EB, Llinas RH, Hillis AE, Gottesman RF. Hemorrhagic transformation in patients with acute ischaemic stroke and an indication for anticoagulation. European journal of neurology: the official journal of the European Federation of Neurological Societies. 2013;20(6):962-7. doi:10.1111/ene.12126.
33 Goyal, M. et al. Endovascular thrombectomy after large-vessel ischaemic stroke: a meta-analysis of individual patient data from five randomised trials. Lancet 387, 1723-1731, doi:10.1016/S0140-6736(16)00163-X (2016).
34 Doberstein, K. et al. L1-CAM expression in ccRCC correlates with shorter patients survival times and confers chemoresistance in renal cell carcinoma cells. Carcinogenesis 32, 262-270, doi:10.1093/carcin/bgq249 (2011).
35 Luby, M. et al. Stroke Mismatch Volume with the Use of ABC/2 Is Equivalent to Planimetric Stroke Mismatch Volume. AJNR. American journal of neuroradiology 34, 1901-1907, doi:10.3174/ajnr.A3476 (2013).
36 Neeb, L. et al. Adapting the computed tomography criteria of hemorrhagic transformation to stroke magnetic resonance imaging. Cerebrovascular diseases extra 3, 103-110, doi:10.1159/000354371 (2013).

1. A method of analyzing a sample from a subject, comprising the steps of:
obtaining a biological sample comprising at least one vesicle from the subject,
measuring the level of one or more biomarkers in the biological sample, and
comparing the level of the one or more biomarkers in the biological sample to a control level of the one or more biomarkers in a control biological sample.

2. The method of claim 1, wherein the at least one vesicle is a brain-derived vesicle.

3. The method of claim 2, wherein the at least one brain-derived vesicle comprises neuron-derived extracellular vesicles (NDEs), astrocyte-derived extracellular vesicles (ADEs), and oligodendrocyte-derived extracellular vesicles (ODEs).

4. The method of claim 1, wherein at least one of the one or more biomarkers comprise CD9, L1CAM, CD171, EAAT1, EAAT2, and MOG.

5. The method of claim 1, wherein the biological sample and the control biological sample comprise whole blood, serum plasma, cerebral spinal fluid, or brain tissue.

6. A method of analyzing a sample from a subject to determine hemorrhagic transformation, comprising the steps of:
obtaining a biological sample comprising at least one vesicle from the subject,
measuring the level of the at least one vesicle in the biological sample, and
comparing the level of the at least one vesicle in the biological sample to a control level of the at least one vesicle in a control biological sample, wherein an increase in the at least one vesicle in the biological sample as compared to the control level of the at least one vesicle in a control biological sample is an indicator of hemorrhagic transformation after a stroke in a patient.

7. The method of claim 6, wherein the at least one vesicle is a brain-derived vesicle.

8. The method of claim 6, wherein hemorrhagic transformation comprises brain hemorrhage, intracranial hemorrhage, or a combination thereof.

9. A post-stroke blood-brain barrier assay, wherein the assay comprises the steps of:
obtaining a biological sample comprising at least one vesicle from the subject,
measuring the level of one or more biomarkers in the biological sample, and
comparing the level of the one or more biomarkers in the biological sample to a control level of the one or more biomarkers in a control biological sample.

10. The assay of claim 9, wherein the at least one vesicle comprises neuron-derived extracellular vesicles (NDEs), astrocyte-derived extracellular vesicles (ADEs), and oligodendrocyte-derived extracellular vesicles (ODEs).

11. The assay of claim 9, wherein at least one of the one or more biomarkers comprise CD9, L1CAM, CD171, EAAT1, EAAT2, and MOG.

12. The assay of claim 11, wherein the at least one vesicle comprises a brain-derived vesicle.

13. The assay of claim 9, wherein the biological sample and the control biological sample comprise whole blood, serum plasma, or brain tissue.

14. A post-stroke blood-brain barrier assay, wherein the assay comprises the steps of:
obtaining a biological sample comprising at least one vesicle from the subject,
measuring the level of the at least one vesicle in the biological sample, and
comparing the level of the at least one vesicle in the biological sample to a control level of the at least one vesicle in a control biological sample, wherein an increase in the at least one vesicle in the biological sample as compared to the control level of the at least one vesicle in a control biological sample is an indicator of hemorrhagic transformation after a stroke in a patient.

15. The assay of claim 14, wherein the at least one vesicle comprises neuron-derived extracellular vesicles (NDEs), astrocyte-derived extracellular vesicles (ADEs), and oligodendrocyte-derived extracellular vesicles (ODEs).

16. The assay of claim 14, wherein at least one of the one or more biomarkers comprise CD9, L1CAM, CD171, EAAT1, EAAT2, and MOG.

17. The assay of claim 16, wherein the at least one vesicle comprises a brain-derived vesicle.

18. The assay of claim 14, wherein the biological sample and the control biological sample comprise whole blood, serum plasma, or brain tissue.

19. A method comprising:
a) obtaining a biological sample comprising vesicles from a subject;
b) isolating vesicles from the biological sample; and
c) detecting one or more biomarkers from the isolated vesicles, wherein at least one of the one or more biomarkers are selected from the group consisting of CD9, L1CAM, CD171, EAAT1, EAAT2, and MOG.

20. The method of claim 19, wherein the vesicles comprise at least one brain-derived vesicle.

21. The method of claim 19, wherein the at least one brain-derived vesicle comprises neuron-derived extracellular vesicles (NDEs), astrocyte-derived extracellular vesicles (ADEs), and oligodendrocyte-derived extracellular vesicles (ODEs).

22. The method of claim 19, wherein the biological sample and the control biological sample comprise whole blood, serum plasma, or brain tissue.

23. A method of analyzing a sample from a subject to determine hemorrhagic transformation, comprising the steps of:
obtaining a biological sample comprising at least one vesicle from the subject,
measuring the level of one or more biomarkers in the biological sample,
preparing a control biological sample that comprises vesicles; and
comparing the level of the one or more biomarkers in the biological sample to a control level of the one or more biomarkers in the control biological sample,
wherein an increase in the one or more biomarkers in the biological sample as compared to the control level of the one or more biomarkers in a control biological sample is an indicator of hemorrhagic transformation after a stroke in a patient.

24. The method of claim 23, wherein at least one of the one or more biomarkers comprise CD9, L1CAM, CD171, EAAT1, EAAT2, and MOG.

25. The method of claim 23, wherein hemorrhagic transformation comprises brain hemorrhage, intracranial hemorrhage, or a combination thereof.

26. The method of claim 23, wherein the vesicles comprise at least one brain-derived vesicle.

27. A method of analyzing a sample from a subject to determine hemorrhagic transformation, comprising the steps of:
obtaining a biological sample comprising at least one vesicle from the subject,
measuring the level of one or more biomarkers in the biological sample,
preparing a control biological sample that comprises neural cell type vesicles; and
comparing the level of the one or more biomarkers in the biological sample to a control level of the one or more biomarkers in the control biological sample,
wherein an increase in the one or more biomarkers in the biological sample as compared to the control level of the one or more biomarkers in a control biological sample is an indicator of hemorrhagic transformation after a stroke in a patient.

28. The method of claim 27, wherein at least one of the one or more biomarkers comprise CD9, L1CAM, CD171, EAAT1, EAAT2, and MOG.

29. The method of claim 27, wherein hemorrhagic transformation comprises brain hemorrhage, intracranial hemorrhage, or a combination thereof.

30. The method of claim 27, wherein the vesicles comprise at least one brain-derived vesicle.
